(19) United States
(12) Patent Application Publication
(10) Pub. No.: US 2009/0010801 A1
(43) Pub. Date: Jan. 8, 2009

(54) AIR CLEANER

(76) Inventors: Oliver J. Murphy, Bryan, TX (US); Brian Hennings, College Station, TX (US); Anthony J. Ragucci, Bryan, TX (US); Anuncia Gonzalez-Martin, San Jose, CA (US)

Correspondence Address: STREETS & STEELE, 13831 NORTHWEST FREEWAY, SUITE 355, HOUSTON, TX, 77040, US

(21) Appl. No.: 12/120,779

(22) Filed: May 15, 2008

Related U.S. Application Data
(60) Provisional application No. 60/938,077, filed on May 15, 2007.

(51) Int. Cl. A61L 9/00; B01D 53/02; B01D 50/00; B03C 3/017; B01D 53/00

(52) U.S. Cl. 422/4; 422/122

(57) ABSTRACT

Apparatus and method for cleaning air. An air cleaner includes a housing that defines an airflow pathway and a catalytic reactor having a catalyst secured on a porous substrate that is disposed transverse to the airflow pathway. Preferably, the catalyst includes a light activated oxidizing photocatalyst or a thermally activated oxidizing catalyst. A photocatalytic reactor will include a light source directed at a light activated oxidizing photocatalyst, such as TiO₂ particles or a binary oxide particle species, which is disposed on the porous substrate. Most preferably, a metal catalyst is disposed on the photocatalyst particles at a concentration or loading between about 0.01 wt % and about 5 wt %. The air cleaner may further comprise an adsorption matrix upstream of the catalytic reactor, optionally in combination with a heater. A particulate filter and/or an electrostatic precipitator may also be disposed upstream of the adsorption matrix and the catalytic reactor.

### CROSS-REFERENCE TO RELATED APPLICATIONS

This application claims priority of U.S. Provisional Application No. 60/938,077 filed on May 15, 2007.

### BACKGROUND OF THE INVENTION

1. Field of the Invention

The present invention relates to air cleaning systems that remove particulates, volatile organic compounds (VOCs), unwanted or toxic vapors, bio-aerosols and other undesirable matter in the air.

2. Description of the Related Art

A variety of undesirable matter finds its way into the indoor air that we breathe. Exposure to allergens from plants and animals can cause allergy symptoms in a large segment of the population. Dust and particulates in the air can also cause allergy symptoms, but may also form an unsightly accumulation throughout an environment and interfere with the proper operation of electronic devices. Some sources of dust and particulates are found indoors, such as a clothes drier, and outdoor dust and particulates come indoors through doors, windows and on clothes. Chemical vapors can arise from the use of household cleaners, off-gassing of new paint or carpet, and various other sources. Such vapors can cause odors and headaches and may be linked to other medical conditions. Even smoke and soot are emitted in small quantities from candles, ovens and stoves. Airborne bacteria, viruses, cysts and spores may also be present.

The variety of matter that can be in the air complicates the task of cleaning the air. Conventional particulate filters and even high efficiency particulate air (HEPA) filters are not designed to combat or remove vapors and extremely fine particulates, such as less than 0.3 micrometers. Additionally, conventional particulate filters, including HEPA filters, have a high pressure drop across them requiring large air movers that consume a significant amount of energy. Electrostatic precipitators do not have an inherent limitation regarding the size of particulates that can be removed and are easier to clean, but are similarly unable to remove vapors, including volatile organic compounds (VOCs). Furthermore, electrostatic precipitators produce a limited amount of ozone gas. Although this ozone may in fact reduce odors, it is generally desirable to remove the ozone from the air prior to exhausting the air from the cleaner.

In order to remove unwanted or toxic vapors from the air, existing indoor cleaner systems have relied upon an adsorption matrix to extract and secure the vapors. The adsorption matrix eventually becomes saturated and must be discarded. While the consumable adsorption matrix is undoubtedly effective in removing vapors from the air, the user must go to the time and expense of installing a new adsorption matrix and the used matrix potentially becomes a mixed waste creating significant disposal issues.

Complex air cleaner systems have been developed with independent stages or unit processes that are each designed to address one aspect of cleaning the air. Operating these stages in series can produce the cumulative effect of removing the various forms of undesirable matter from the air. However, some stages require periodic cleaning and other stages require periodic replacement. As a result, consumer diligence is required in order to maintain the air cleaner in the condition necessary to continue cleaning the air in a proper manner.

For example, U.S. Pat. No. 6,176,977 describes an electro-kinetic electrostatic air conditioner including a self-contained ion generator that provides air with ions and ozone. The ion generator includes a high voltage pulse generator whose output pulses are coupled between one or more wire electrodes spaced apart from “U” shaped collector electrodes. An electric field produced by the high voltage pulses between the arrays produces an electrostatic flow of ionized air containing ozone. Dust and other particulate matter attaches electrostatically to the collector electrodes and the output air is substantially clean of the particulate matter. However, this air conditioner does not provide any way to remove vapors or the ozone produced by the electrodes.

U.S. Pat. No. 7,014,686 describes an air cleaner electrode assembly having an elongated collector electrode and a plurality of elongated discharge electrodes arranged around the collector electrode. A fan moves air in a direction parallel to the length of the electrodes. Particulates in the moving air are given a charge by the discharge electrodes, causing the particulates to be attracted to the collector electrode and become deposited on its surface, a process known as “precipitation.” The clean air is then drawn through the fan and expelled from the housing.

U.S. Patent Publication 2006/0130657 describes a tower ionizer air cleaner including a tower chassis with airflow inlet openings, airflow outlet openings substantially opposite the airflow inlet openings, and an ionizer element positioned within the tower chassis. One or more fan units are positioned substantially vertically within the tower to provide airflow between the inlets and the outlets through the ionizer element.

U.S. Patent Publication 2006/0159594 describes an air sanitizer including a housing, an emitter electrode configured within the housing, a collector electrode within the housing downstream of the emitter electrode, and a germicidal lamp that emits germicidal light within the housing. Accordingly, the air sanitizer is directed at removing particulates and germs from the air.

U.S. Patent Publication 2006/0162564 describes an air cleaner having a prefilter at an inlet end of an air duct, an electrostatic precipitator positioned in the air duct downstream of the prefilter, a post-filter positioned in the air duct downstream of the electrostatic precipitator, and a fan unit positioned in the air duct downstream of the post-filter. The prefilter is described as being rather coarse to avoid impeding the incoming airflow. The post-filter is described as comprising any manner of desired filter element, such as a HEPA filter, an allergen air filter, an electrostatic air filter, charcoal filter, or an anti-microbial filter. Alternatively, the post-filter can include an odor filtration element including some manner of carbon, zeolite, or potassium permanganate filter.

### BRIEF SUMMARY OF THE INVENTION

The present invention provides improved air cleaner systems that remove particulates, volatile organic compounds (VOCs), other unwanted or toxic vapors including ozone, carbon monoxide (CO), oxides of nitrogen (NOₓ), ammonia (NH₃), hydrogen sulfide (H₂S), combustible gases, such as hydrogen (H₂) and natural gas (CH₄), and bio-aerosols from air. Certain embodiments of the air cleaner systems involve fewer components or stages so that the design of such systems can be simplified, consume less space, or increase their capacity. Some embodiments of the air cleaner systems include self-cleaning or regenerable stages that the consumer does not have to replace. Still further embodiments of the air cleaner systems include sensors and control systems to verify proper operation of the air cleaner.

An air cleaner of the present invention comprises a housing that defines an airflow pathway between an air inlet and an air outlet, and a catalytic reactor having a catalyst secured on a porous substrate that is disposed transverse to the airflow pathway to cause air to flow through the porous substrate and into contact with the catalyst. Preferably, the catalytic reactor includes a catalyst selected from the group consisting of a light activated oxidizing photocatalyst and a thermally activated oxidizing catalyst. In embodiments where the catalytic reactor is a photocatalytic reactor, the reactor will include a light source directed at a light activated oxidizing photocatalyst that is disposed on the porous substrate. The light source preferably includes a bulb that produces light selected from ultraviolet light, visible light, and combinations thereof. The oxidizing photocatalyst may comprise TiO₂ particles, or a binary oxide particle species selected from TiO₂/SiO₂, TiO₂/ZrO₂, TiO₂/SnO₂, TiO₂/WO₃, TiO₂/MoO₃, TiO₂/V₂O₅ and combinations thereof. The photocatalyst particle surfaces may also include a catalyst selected from the group consisting of a metal, metal oxide, or metal alloy, Pt group metals, Au group metals, Ir, Ru, Sn, Os, Mo, Zr, Cu, Nb, Rh, Pt—Sn, Pt—Mo, Pt—Ru, Ni—Zr, Pt—Rh, Pt—Ir, Pt—Ru—W, Pt—Ru—Os, Pt—Ru—Sn, Pt—Ni—Ti, Pt—Ni—Zr, Pt—Ni—Nb, platinum group metal oxides, gold group metal oxides, tin oxides, tungsten oxides, iridium oxides, rhodium oxides, ruthenium oxides and mixtures thereof. Most preferably, the catalyst disposed on the surfaces of the photocatalyst particles is a metal catalyst provided at a concentration or loading between about 0.01 wt % and about 5 wt %.

The air cleaner may further comprise an adsorption matrix disposed transverse to the airflow pathway. Furthermore, the air cleaner may include a heater in thermal communication with the adsorption matrix. Still further, the catalytic reactor may include a thermally activated oxidizing catalyst and a heater in thermal communication with the oxidizing catalyst, wherein the catalytic reactor is disposed downstream of the adsorption matrix.

In some embodiments, the air cleaner includes a particulate filter and/or an electrostatic precipitator disposed upstream of the adsorption matrix and the catalytic reactor. An exemplary electrostatic precipitator comprises one or more discharge electrode and one of more collection electrode, wherein the one or more collection electrode is a macroporous member disposed transverse to the airflow pathway to cause air to flow through the macroporous member. Preferably, the one or more discharge electrode is negatively charged and the one or more collection electrode is positively charged. The one or more collection electrode optionally comprises two or more layers, wherein the two or more layers have openings that decrease in size from one layer to the next in the direction from the air inlet to the air outlet. In a further embodiment, the one or more collection electrode is secured in a unitary structure with the porous substrate of the catalytic reactor, preferably with the photocatalyst being electrically insulated from the one or more collection electrode.

The present invention also provides a method of removing volatile organic compounds from an air stream. One embodiment of the method includes flowing the air stream through a porous adsorption matrix upstream of a porous oxidizing catalytic reactor, wherein the porous oxidizing catalytic reactor comprises an oxidizing catalyst surface. The volatile organic compounds are adsorbed from the air stream onto the porous adsorption matrix. Periodically, the porous adsorption matrix is heated to revolatilize the adsorbed compounds into the air stream. The air stream and revolatilized compounds are made to flow into the oxidizing catalytic reactor where the revolatilized compounds are oxidized in the presence of an oxidant in the air stream at the oxidizing catalyst surface. The flow rate of the air stream through the catalytic reactor is preferably reduced during the step of periodically heating to an air flow rate that is lower than the air flow rate during the step of adsorbing the volatile organic compounds. By way of example, the oxidant source may be selected from the group consisting of oxygen in air, ozone, vapor phase hydrogen peroxide, and combinations thereof.

In one embodiment of the method, the catalytic reactor includes a thermally activated oxidizing catalyst, wherein the thermally activated oxidizing catalyst is heated to a light off temperature substantially only immediately before and during the step of periodically heating the porous adsorption matrix. In response to completing the step of periodically heating the porous adsorption matrix, the thermally activated oxidizing catalyst is allowed to cool to ambient temperature.

In another embodiment of the method, the catalytic reactor includes a light activated oxidizing catalyst, wherein radical species are photogenerated by directing light photons onto the surface of the oxidizing photocatalyst, and the volatile organic compounds are oxidized in the air stream that contact the radical species. Preferably, the method will serve to inactivate microorganisms in the air stream that contact the radical species.

Other embodiments, aspects, and advantages of the invention will be apparent from the following description and the appended claims.

### BRIEF DESCRIPTION OF THE SEVERAL VIEWS OF THE DRAWINGS

FIG. 1 is a perspective view of a prior art air cleaner system including a plurality of air cleaning stages.

FIG. 2 is a perspective view of an air cleaner system including a photocatalytic stage.

FIG. 3 is a perspective view of an air cleaner system having electrostatic collection electrodes that are transversely oriented porous metal plates.

FIGS. 4A, 4B, and 4C are perspective views of air cleaner systems including various unitized member configurations having both a collection electrode and a photocatalyst layer.

FIGS. 5A, 5B, and 5C are perspective views of air cleaner systems including an adsorption member with a heating element.

FIG. 6 is a perspective view of an air cleaner system having gas sensors disposed in the air intake and air exhaust.

### DETAILED DESCRIPTION OF THE INVENTION

The present invention provides air cleaner systems for improving indoor air quality in residential, commercial and industrial buildings, air in other confined spaces such as in airplanes, automobiles, trains, boats, ships, and spacecraft by removing particulates, oxidizing volatile organic compounds, oxidizing other unwanted, toxic or combustible vapors, including carbon monoxide (CO), oxides of nitrogen (NOₓ), ammonia (NH₃), hydrogen sulfide (H₂S), hydrogen (H₂) and natural gas (CH₄) and destructing ozone as well as inactivating airborne bacteria, viruses, cysts, and spores. The invention achieves these functions through various air cleaner embodiments. None of the prior art air cleaners, discussed above, disclose or suggest that volatile organic compounds, unwanted, toxic or combustible vapors, or airborne bacteria, viruses, cysts and spores should or could be oxidized, destroyed or inactivated rather than simply adsorbed.

The present air cleaner systems remove particulates using an optional prefilter and an electrostatic precipitator. If a prefilter is used, it is preferably a coarse filter that will trap only large, visible particulates such as carpet fiber, cat hair, or other lint. The electrostatic precipitator is preferably downstream of the prefilter and includes one or more discharge electrodes coupled to the negative terminal of a voltage source and one or more collector electrodes coupled to the positive terminal of the voltage source. Electrostatic precipitators of this type are described in U.S. Pat. Nos. 6,176,977 and 7,014,686; U.S. Publication Nos. 2006/0159594; and 2006/0162564; which patents and publications are incorporated by reference herein.

Alternatively, the electrostatic precipitator may include a macroporous collector electrode that extends transversely across the airflow pathway such that the air must pass through the openings of the collector electrode. Suitable material for a macroporous collector electrode include, without limitation, electronically conductive screens, foams, packed beds, or combinations thereof. The macroporous collector electrode may include a single uniform layer or a plurality of layers. If a plurality of macroporous layers is used, they are preferably arranged to provide graduated porosity with the most upstream layer having the largest pores or passageways and the most downstream layer having the smallest pores or passageways.

The present air cleaner systems oxidize volatile organic compounds or other unwanted, toxic or combustible vapors using a catalytic reactor, preferably located downstream of the one or more particulate removal elements. The catalytic reactor may provide a light activated photocatalyst oxidizing surface facing an ultraviolet and/or visible light source or take the form of a thermally activated oxidizing catalyst as might be used in a catalytic converter. A preferred photocatalyst oxidizing surface is prepared by coating a photocatalyst onto a porous substrate, most preferably an electrically grounded porous substrate. Preferably, the photocatalyst-coated porous substrate is positioned transverse to the airflow so that the air must pass through the pores of the substrate. The volatile organic compounds, other unwanted, toxic or combustible vapors, or airborne pathogenic microorganisms in the air are made to pass in close proximity to the photocatalyst oxidizing surface where photogenerated radical species that are capable of oxidizing, destroying, or inactivating the compounds, vapors, or microorganisms are being formed.

The photocatalytic element oxidizes organic and inorganic contaminants in air by combining a semiconductor photocatalyst surface, an oxidant source, water vapor and a UV and/or visible light source directed onto the surface of the photocatalyst, where preferably the photocatalyst is comprised of small, high surface area particles. The semiconductor material is preferably an n-type semiconductor and more preferably an n-type metal oxide semiconductor. Organic contaminants, water vapor and oxygen in the air are delivered through the porous substrate to the surface of the supported photocatalyst particles. In addition to the oxygen in the air, either ozone gas and/or vapor phase hydrogen peroxide, produced internally or externally of an air cleaner system of the present invention can be used as an oxidant source. Efficient photocatalytic oxidation of organic and inorganic compounds occurs on a porous n-type semiconductor photocatalytic surface, such as provided by supported TiO₂ particles, nitrogen-doped TiO₂ particles, or binary oxide particles selected from TiO₂/SiO₂, TiO₂/ZrO₂, TiO₂/SnO₂, TiO₂/WO₃, TiO₂/MoO₃, and TiO₂/V₂O₅. In a preferred embodiment, the photocatalytic surface of the n-type semiconductor material has very fine catalyst particles disposed thereon. The catalyst can be a metal, metal oxide, or metal alloy, such as Pt group metals, Au group metals, Ir, Ru, Sn, Os, Mo, Zr, Cu, Nb, Rh, Pt—Sn, Pt—Mo, Pt—Ru, Ni—Zr, Pt—Rh, Pt—Ir, Pt—Ru—W, Pt—Ru—Os, Pt—Ru—Sn, Pt—Ni—Ti, Pt—Ni—Zr, Pt—Ni—Nb, platinum group metal oxides, gold group metal oxides, tin oxides, tungsten oxides, iridium oxides, rhodium oxides, ruthenium oxides and mixtures thereof. The preferred concentration or loading of metal catalyst particles supported on the surfaces of the n-type semiconductor photoactive particles is between about 0.01 wt % and about 5 wt % metal catalyst.

The photoactive, n-type semiconductor material, or photocatalyst, is preferably a porous titanium dioxide layer or surface formed on a porous substrate. The substrate may be made from virtually any material that is sufficiently porous to pass an air stream therethrough, can provide physical support for the photocatalyst material and is resistant to chemical or thermal degradation. For example, the substrate could be made from a porous metal, porous carbon or graphite, a sintered porous glass or a porous ceramic and could be in the form of a foam, a mesh, or a cloth, either woven or unwoven. The photocatalyst, which may consist of the photoactive, n-type semiconductor material alone, or the catalyzed photoactive, n-type semiconductor material, may be applied to the porous substrate by any means including: (1) applying a solution or slurry with a brush followed by sintering; (2) forming a sol-gel, applying the sol-gel by spraying, dipping, or spin coating, then drying and curing; (3) vacuum deposition processes, such as chemical vapor deposition and physical vapor deposition; or (4) electrochemical oxidation of a porous metal in an aqueous solution. The term “porous” as used in reference to the photocatalyst surface is intended to include any photocatalyst surface having passages therethrough for the stream of air to be treated. Therefore, the photocatalyst layer itself may be porous or, conversely, the photocatalyst may be a dense layer that simply leaves the pores of the substrate open. Theoretically, if the semiconductor photocatalyst material had sufficient strength and appropriate pore size, the porous substrate would not be necessary.

Alternatively, the catalytic reactor may take the form of a thermally activated oxidizing catalyst supported on a substrate. Preferably, the oxidizing catalyst may be a two-way or three-way catalyst composition as would be found in an automotive catalytic converter, which may be supported on a monolith, ceramic or metallic structure. However, in order to effectively oxidize volatile organic compounds, other unwanted, toxic or combustible vapors, and airborne pathogenic microorganisms, the oxidizing catalyst may require heating to a light off temperature. Such heating can be provided by introducing hydrogen gas along with the already present oxygen gas in the air, or by use of an electrical heating element. Because of the elevated temperatures necessary for the oxidizing catalyst to operate, and the associated undesirable heating of the air stream to be treated, this type of catalytic reactor is preferably not operated on a continuous basis. Rather, such an embodiment is more appropriate for periodic operation to oxidize high concentrations of volatile organic compounds and other unwanted or combustible vapors, as will be described in more detail below.

The present air cleaner systems may further include a regenerable adsorption matrix. An adsorption matrix, such as a porous layer comprising activated carbon, charcoal, alumina, silica or zeolites, is preferably disposed downstream of the filter elements and preferably disposed transversely across the path of the airflow to cause contact with the air. Volatile organic compounds, together with other unwanted or combustible vapors, as well as airborne pathogenic microorganisms, are adsorbed out of the flowing air stream and into the adsorption matrix. The adsorption of VOCs and other unwanted species will continue until the matrix is saturated. However, a heater element is disposed in thermal communication with the adsorption matrix in order to periodically drive off or revolatilize the compounds or species out of the adsorption matrix and into the air. By positioning the regenerable adsorption matrix upstream of the oxidizing catalytic reactor, the VOCs, other unwanted or combustible vapors, or airborne pathogenic microorganisms are oxidized, destroyed or inactivated.

The adsorption matrix conveniently draws the VOCs, other unwanted or combustible vapors, or airborne pathogenic microorganisms, out of the air on a substantially continuous basis, but has a finite capacity. Periodically regenerating the adsorption matrix restores the adsorption matrix to its original capacity and avoids the otherwise necessary steps of discarding the saturated adsorption matrix and replacing it with a fresh adsorption matrix. Since the regeneration can occur periodically, it is not necessary to continuously operate the oxidizing catalytic reactor. Rather, the VOCs, other unwanted or combustible vapors, or airborne pathogenic microorganisms, can be oxidized, destroyed or inactivated by operating the catalytic reactor during the regeneration of the adsorption matrix. If the oxidizing catalytic reactor in the air cleaner is a light activated photocatalyst material, then it may be desirable to operate the oxidizing catalyst even when the adsorption matrix is not being regenerated in order to inactivate bacteria, viruses, cysts, and spores and also to oxidize any VOCs that pass through the adsorption matrix. However, if the catalytic reactor is a thermally activated oxidizing catalyst that must operate at a light off temperature, then it is preferable to operate the catalytic reactor primarily or only during the regeneration process when a higher concentration of VOCs, other unwanted or combustible vapors, or airborne pathogenic microorganisms, is provided. An optional thermoelectrically cooled heat exchanger may be used during the high temperature oxidation process in order to remove heat added to the flowing air stream and avoid heated air exiting the air cleaner.

The porous adsorption matrix may, for example, be made from activated carbon, carbon fibers, carbon powder, carbon granules, charcoal, single-walled or multi-walled carbon nanotubes, buckminsterfullerenes or “buckyballs” such as C₆₀, small particles of metal oxides (such as magnesium oxide, calcium oxide, aluminum oxide, or silicon oxide), or combinations thereof. The fibers, powders, granules, nanotubes, and particles of various materials may be mixed together in optimum ratios and may be held or bonded together by a cured binding agent. Binding agents well known to one skilled in the art such as an organic polymer, an inorganic glass material, or an inorganic binder, such as aluminum phosphate, other cements, mortars, and the like may be used. Optionally, the individual components, or mixtures of them may be impregnated into, or supported on macroporous carbon cloth or carbon felt, polymer cloth, polymer felt, or polymer foam having open cells, silica cloth, alumina cloth, or other ceramic based cloths or foams.

The preferred adsorption reagents are composites comprising finely divided particles of a first metal oxide selected from MgO, CaO, Al₂O₃, SnO₂, TiO₂ and mixtures thereof, these particles being at least partially coated with a second metal oxide selected from Fe₂O₃, Cu₂O, NiO, CoO and mixtures thereof. These composites most preferably comprise between 90 and 99 percent of the first metal oxide. Metal oxide or metal hydroxide adsorbents may be used alone or in combination, such as those adsorbents selected from MgO, CeO₂, CaO, TiO₂, ZrO₂, FeO, V₂O₅, V₂O₃, Mn₂O₃, Fe₂O₃, CuO, NiO, ZnO VAl₂O₃, SnO₂, Ag₂O, SrO, BaO, Mg(OH)₂, Ca(OH)₂, Al(OH)₃, Sr(OH)₂, Ba(OH)₂, Fe(OH)₃, Cu(OH)₃, Ni(OH)₂, Co(OH)₂, Zn(OH)₂, and AgOH. Most preferably, these adsorbents are powders prepared by aerogel techniques. Optionally, the adsorbents may have reactive atoms (such as chlorine, bromine or iodine) stabilized on their surfaces, species adsorbed on their surfaces, or coated with a second metal oxide.

Iron oxide-magnesium oxide composites are examples of finely divided composite materials that may be included in the adsorption element in order to destroy chlorinated hydrocarbons (chlorocarbons) and chlorofluorocarbons. Preferably, the composites comprise a first metal oxide, such as MgO, coated with a thin layer of a transition metal oxide, such as Fe₂O₃. Materials and applications such as these are described in U.S. Pat. No. 5,712,219, which is incorporated by reference herein.

In a further embodiment of the invention, two or more of the elements in the air cleaner are combined into a unitary structure. Using a unitary structure reduces the number of separate parts and correspondingly reduces the number of independent support structures in the housing. For example, a generally transverse porous conductive screen or foam may form a unitary structure with a catalytic reactor or a catalyzed substrate, such as a light activated photocatalyst coated porous substrate. The porous conductive screen or foam would form the collector electrode of an electrostatic precipitator and a first face of it would be placed opposite the discharge electrodes. The light activated, porous, photocatalyst coated second face would serve to oxidize VOCs, and other unwanted or combustible vapors, or airborne pathogenic microorganisms, and would be placed opposite a UV and/or visible light source. Preferably, the unitary structure will also include a porous polymeric or ceramic insulator layer disposed between the collector electrode and the photocatalyst coated porous substrate. While these structures could be made unitary by securing each layer at the edges, it is preferable to intimately secure the layers along the interfaces there between, such as by intimately forming one porous layer upon the other or by use of adhesives. Additional elements of the air cleaner, such as the prefilter, may also be included in the unitary structure as will be described in reference to the figures below.

A unitary structure, as described above, is preferably transverse to the airflow, but may be variously shaped such as planar, arced, pitched, wavy, spiked or corrugated. The shape of the unitary structure is preferably adapted so that the collector electrode cooperates efficiently with the placement of the one or more discharge electrodes and so that the light activated photocatalyst layer cooperates efficiently with the placement of the UV and/or visible light source.

In a still further embodiment, a novel collector electrode structure is introduced. A packed bed of electrically conductive objects, such as metal-coated polymeric or ceramic balls, is retained between porous structures and preferably coupled to the positive terminal of the voltage source so that the packed bed serves as a collector electrode. The resulting three dimensional matrix may have uniform porosity or spacing as would result from using a single size of uniform balls. However, the packed bed may have nonuniform porosity or spacing as the result of using nonuniform sizes or shapes of objects. Optionally, the objects may be used in multiple sizes or shapes and layered within the packed bed to form a gradient of porosity or spacing. A most preferred porosity gradient provides the largest pore size across a front face of the packed bed and the smallest pore size across the back face of the packed bed.

Although airflow through the air cleaner can be induced solely by the electrostatic precipitator, the air cleaner preferably incorporates at least one electric fan or blower to force air through each of the elements or stages. The fan(s) may be disposed at any position within the airflow pathway of the air cleaner housing, such as at the air inlet, at the air outlet or between elements or stages. The housing preferably forms the airflow pathway snugly about the elements or stages so that there is little or no air bypassing the elements or stages.

A suitable ozone destruction catalyst includes manganese dioxide, derivatives of manganese dioxide, mixtures of iron oxide and manganese dioxide, carbon, and palladium or platinum supported on carbon, either supported or unsupported in the form of powders, granules, fibers, cloths, meshes, or foams. Alternatively, ozone adsorbents such as catalyzed or noncatalyzed zeolites may be used.

Carbon monoxide (CO) gas may also be present in the air and can be potentially harmful. Therefore, it may be beneficial to include a CO oxidation catalyst that, in the presence of air or oxygen gas, converts the carbon monoxide to carbon dioxide (CO₂). Exemplary catalysts include gold catalysts supported on metal oxide particles, such as high surface area titanium dioxide powder or tin dioxide powder. Suitable metal oxide-supported catalysts may be prepared by methods selected from co-precipitation, deposition-precipitation, and suspension spray reaction. Exemplary catalysts are described in U.S. Pat. No. 6,616,903.

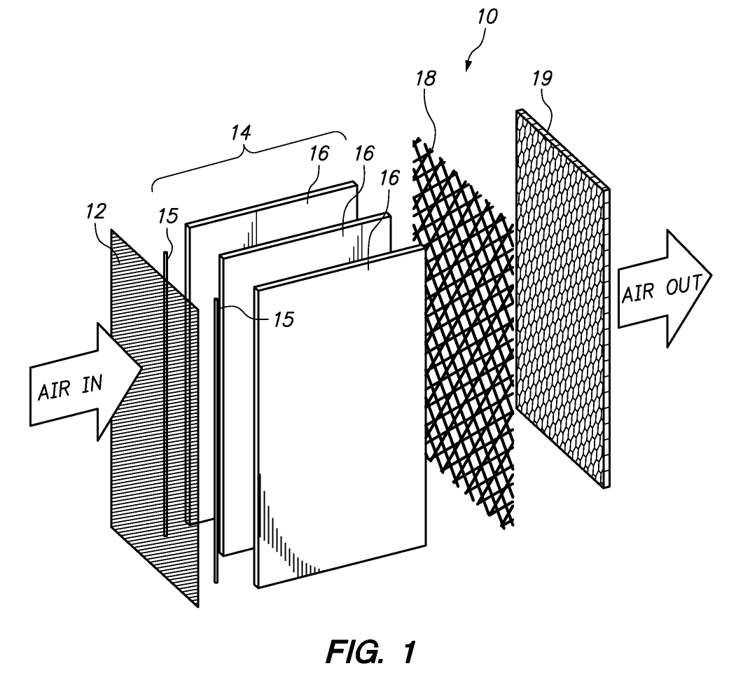

FIG. 1 is a perspective view of a prior art air cleaner system 10 including a plurality of air cleaning stages. The system 10 includes a coarse prefilter 12, an electrostatic precipitator stage 14, a carbon-based VOC adsorber 18, and a porous ozone destruction catalyst coated substrate 19. The electrostatic precipitator stage 14 includes a pair of discharge electrode wires or rods 15 spaced apart from three metal plate collector electrodes 16, wherein the discharge electrodes are negatively charged and the collector electrodes are positively charged. The negatively charged discharge electrodes ionize particles in the air, causing them to be attracted to the positively charged collector electrodes. Movement of the ions between the electrodes can impart airflow to the surrounding air and directs the air through the adsorber and the catalyst coated substrate.

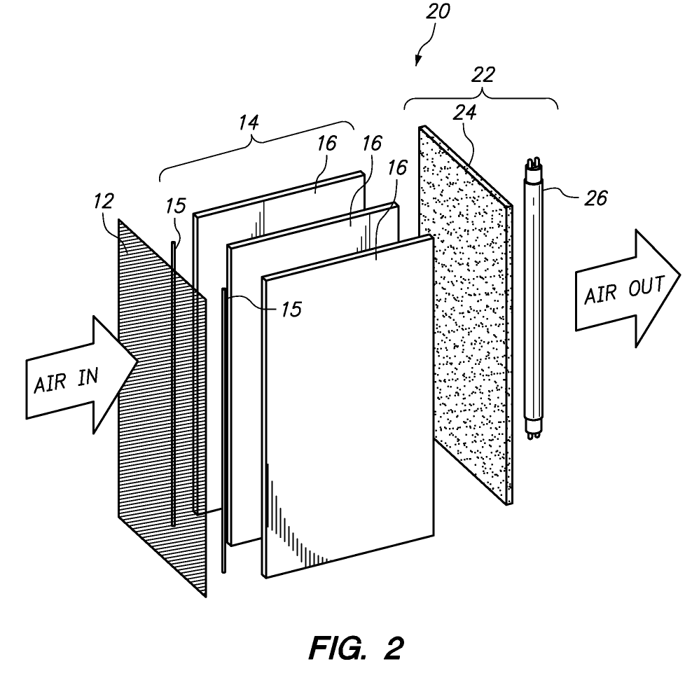

FIG. 2 is a perspective view of an air cleaner system 20 of the present invention including a light activated photocatalytic reactor stage 22. A photocatalyst-coated porous substrate 24 is disposed in the airflow pathway and a UV and/or visible light source 26 is positioned to direct photons of light onto the photocatalyst surface. As air passes through the porous substrate 24, volatile organic compounds (VOCs), other unwanted or combustible vapors, or airborne pathogenic microorganisms, are oxidized on the photocatalyst surface. Accordingly, VOCs, other unwanted or combustible vapors, or airborne pathogenic microorganisms, are eliminated from the air without using a disposable adsorption matrix. The UV and/or visible light source not only supports oxidation on the photocatalyst surface, but may also provide germicidal action.

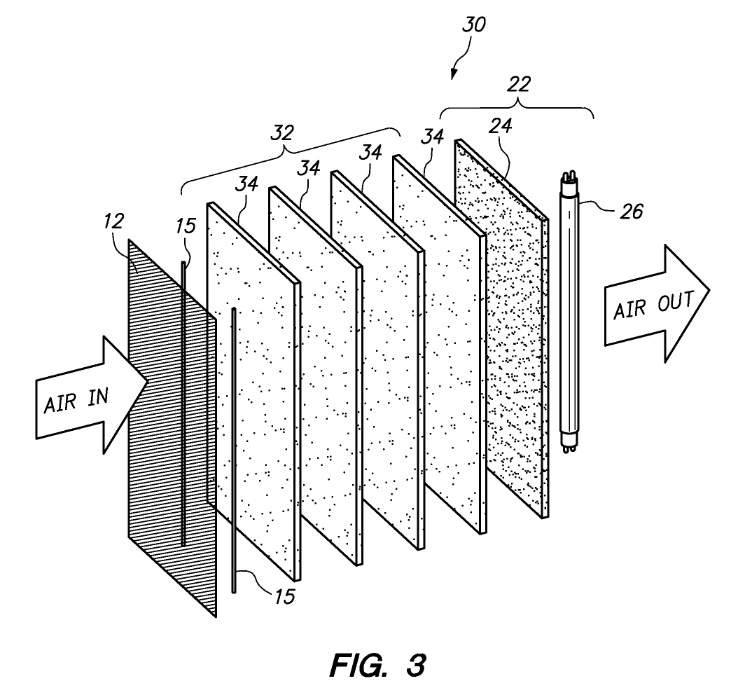

FIG. 3 is a perspective view of an air cleaner system 30 having an electrostatic precipitator 32 with discharge electrode wires 15 and porous collection electrodes 34. The porous collection electrodes 34 are transversely oriented across the airflow pathway. Although the collector electrode may include a single layer of the porous conductive material 34, the collector electrode is shown as multiple layers of the porous conductive material 34. The prefilter 12 and the photocatalytic reactor stage 22 operate in the same manner as in FIG. 2.

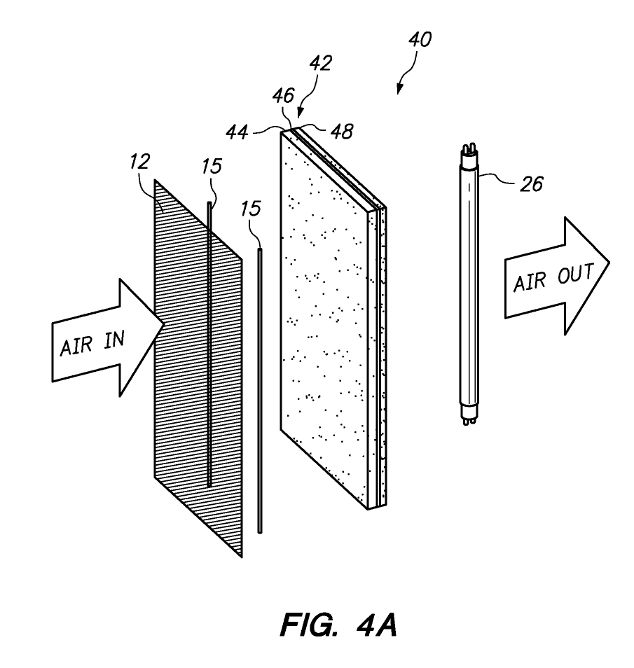

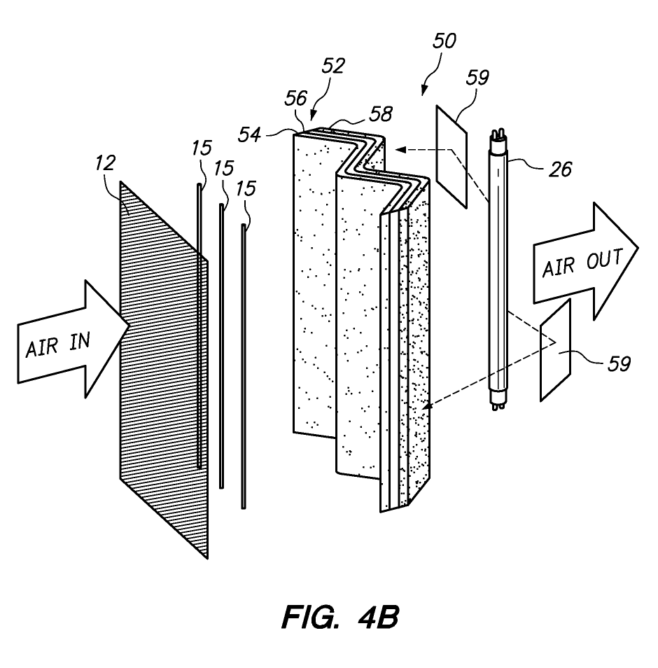

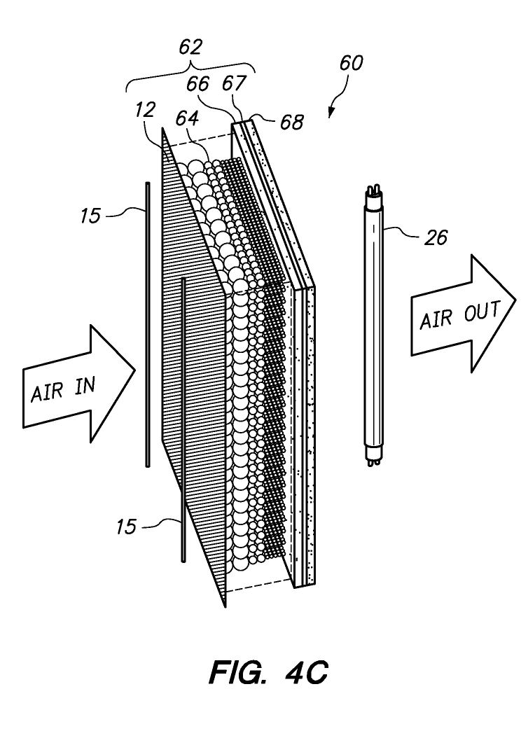

FIGS. 4A, 4B, and 4C are perspective views of air cleaner systems including various unitized member configurations having both a collection electrode and a light activated photocatalyst layer. In FIG. 4A, the air cleaner system 40 includes a unitized member 42 comprising a porous metal screen or foam 44, a porous polymeric or ceramic insulator 46, and a light activated photocatalyst coated porous substrate 48, where preferably the substrate is electrically grounded. The porous metal screen or foam 44 is coupled to a positive terminal of a voltage source and faces the discharge electrode wires 15 that are coupled to a negative terminal of the voltage source in order to cooperatively support electrostatic precipitation. On the other side of the porous polymeric or ceramic insulator 46, the photocatalyst coated porous substrate 48 faces the light source 26 to enable photocatalytic oxidation of volatile organic compounds, other unwanted or combustible vapors, or airborne pathogenic microorganisms, as well as the destruction of ozone that may be produced by the electrostatic precipitator electrodes.

In FIG. 4B, the air cleaner system 50 includes a unitized member 52 comprising a porous metal screen or foam 54, a porous polymeric or ceramic insulator 56, and a light activated photocatalyst coated porous substrate 58, where preferably the substrate is electrically grounded. The unitized member 52 is contoured in a manner that may be described as wavy or corrugated. The porous metal screen or foam 54 is coupled to a positive terminal of a voltage source and faces the discharge electrode wires 15 that are coupled to a negative terminal of the voltage source in order to cooperatively support electrostatic precipitation. On the other side of the ceramic insulator 56, the light activated photocatalyst coated porous substrate 58 is similarly wavy or corrugated and faces the UV and/or visible light source 26 to enable photocatalytic oxidation of volatile organic compounds, other unwanted or combustible vapors, or airborne pathogenic microorganisms, as well as the destruction of ozone that may be produced by the electrostatic precipitator electrodes. One or more mirrored or reflective surfaces 59 (shown schematically and not to scale) may be used to redirect some of the light onto the surface of the photocatalyst substrate 58, most preferably to achieve a substantially uniform coverage. However, the mirrors or reflectors 59 should be configured or positioned to avoid producing a pressure drop in the airflow pathway through the air cleaner system, such as by positioning the mirrors or reflectors along the inner walls of the housing and directly downstream of the light source 26. It may also be possible to coat the rearward facing surfaces of a light bulb to direct all of the light forward at a predetermined spread angle. Furthermore, where the air cleaner system airflow exhaust is covered by a grill, the inside surface facing the light source 26 may be made to reflect light onto the surface of the photocatalyst.

In FIG. 4C, the air cleaner system 60 includes a unitized member 62 comprising the prefilter 12, a packed bed collector electrode 64, a porous metal screen or foam 66, a porous polymeric or ceramic insulator 67, and a light activated photocatalyst coated porous substrate 68, where preferably the substrate is electrically grounded. The porous metal screen or foam 66 and the packed bed collector electrode 64 are coupled to a positive terminal of a voltage source and face the discharge electrode wires 15 that are coupled to a negative terminal of the voltage source in order to cooperatively support electrostatic precipitation. The prefilter 12 may also be positively charged, but is primarily responsible for physically trapping large particulates and retaining the conductive objects within the packed bed 64. However, it is also possible for the porous metal screen or foam 66 and/or the prefilter 12 to serve as electrical distribution grids for the collector electrode 64. On the other side of the porous polymeric or ceramic insulator 67, the light activated photocatalyst coated porous substrate 68 faces the light source 26 to enable photocatalytic oxidation of volatile organic compounds, other unwanted or combustible vapors, or airborne pathogenic microorganisms, as well as the destruction of ozone that may be produced by the electrostatic precipitator electrodes.

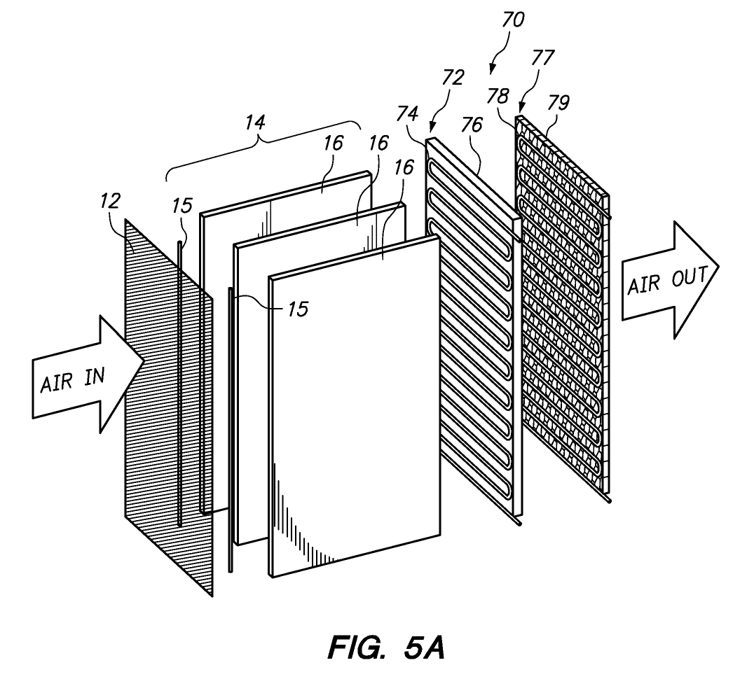

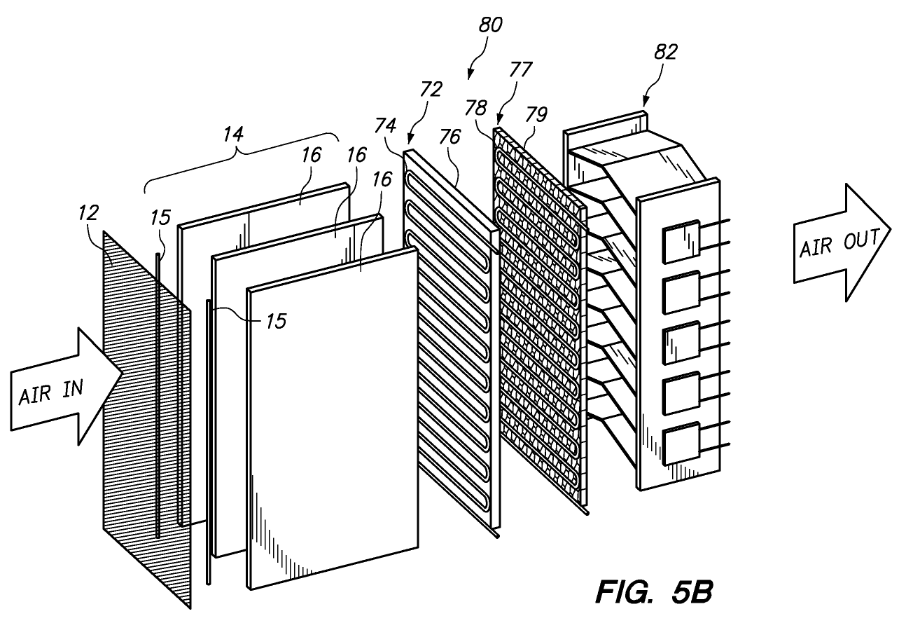

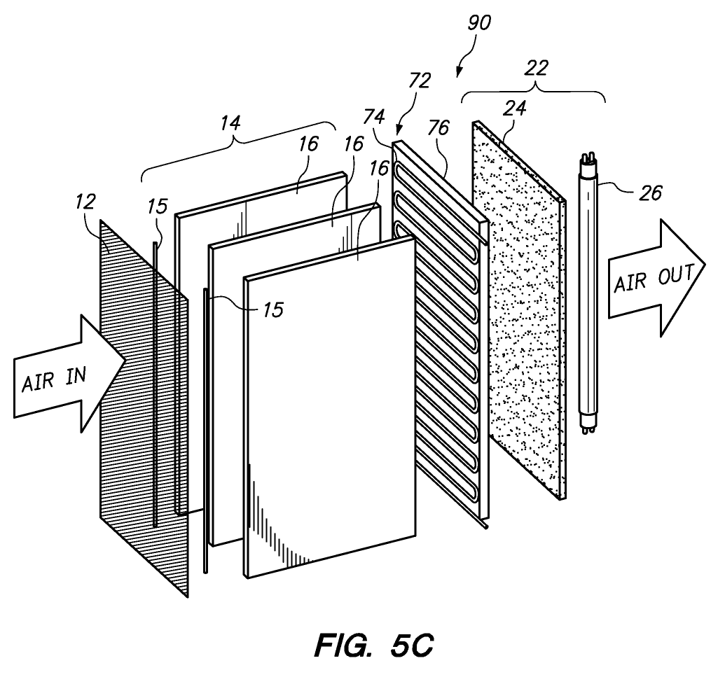

FIGS. 5A, 5B, and 5C are perspective views of air cleaner systems including a regenerable adsorption member with a heating element. In FIG. 5A, the air cleaner system 70 includes the prefilter 12, the electrostatic precipitator 14, a regenerable adsorption member 72, and a thermally activated porous oxidizing catalytic reactor 77. The regenerable adsorption member 72 is made from a porous VOC adsorption matrix 76, such as an activated carbon matrix, in thermal communication with a heater element 74. To maximize the heat transfer from the heater element 74 to the adsorption matrix 76, the heater element may be embedded within the matrix or positioned on the upstream side of the matrix so that air passing through the matrix will convey heat throughout the matrix. The thermally activated porous oxidizing catalyst element 77 includes a catalyst coated substrate 79 and a heater element 78. The heater element 78 is in thermal communication with the catalyst coated substrate 79, preferably via direct contact, in order to heat the catalyst to a light off temperature for oxidizing VOCs. Coordinated operation of the regenerable adsorption matrix 76 and the thermally activated porous oxidizing catalyst element 77 enable the continuous and long term removal of VOCs from the airflow. As previously described, the VOCs are adsorbed onto the adsorption matrix on a substantially continuous basis, but before the capacity of the adsorption matrix 76 is exceeded the adsorption matrix is regenerated. Regeneration of the adsorption matrix involves heating the porous oxidizing catalyst element 77 to a light off temperature, then turning on the heater element 74 to drive the VOCs out of the matrix. As the airflow passes from the adsorption matrix to the oxidizing catalyst element, the VOCs are transferred to the oxidizing catalyst element where the VOCs are oxidized to carbon dioxide and water vapor. The heating elements 78, 74 are then turned off and the adsorption matrix is allowed to adsorb VOCs until the next regeneration.

During the process of regenerating the adsorption matrix, the rate of airflow through the air cleaner system is preferably reduced in order to increase the residence time of the revolatilized VOCs in the oxidizing catalyst element and to minimize heat transfer from the adsorber and the oxidizing catalyst element into the air that will pass through the system exhaust and into the room. Accordingly, the reduced airflow rate improves the operating efficiency of the oxidizing catalyst element while also serving to conserve energy and avoid unnecessarily heating large volumes of air.

In FIG. 5B, the air cleaner system 80 includes the same elements as in system 70 of FIG. 5A, except for the addition of a thermoelectrically cooled heat exchanger 82 on the air outlet side of the thermally activated porous oxidizing catalyst element 77. The heat exchanger is designed to remove a substantial amount of the heat introduced into the flowing air by the heated adsorption member 72 and by the thermally activated porous oxidizing catalyst element 77 during the regeneration cycle.

In FIG. 5C, the air cleaner system 90 includes the same elements as in system 70 of FIG. 5A, except that the thermally activated porous oxidizing catalyst element 77 has been replaced by the light activated photocatalytic reactor stage 22 of FIG. 2. A photocatalyst-coated porous substrate 24 is disposed in the airflow pathway and a UV and/or visible light source 26 is positioned to direct suitably energized photons of light onto the photocatalyst surface. As air passes through the porous substrate 24, volatile organic compounds, other unwanted or combustible vapors, or airborne pathogenic microorganisms, are oxidized on the photocatalyst surface. The photocatalytic reactor stage 22 is preferably operated on a substantially continuous basis, but may optionally operate periodically as part of the adsorption matrix regeneration cycle. In the latter instance, the UV and/or visible light source 26 would only need to be operated while VOCs are being driven out of the adsorption matrix 76 by the heat element 74. The UV component of the light source may not only support oxidation on the light activated photocatalyst surface, but may also provide germicidal action if photons of light having suitable energy are emitted.

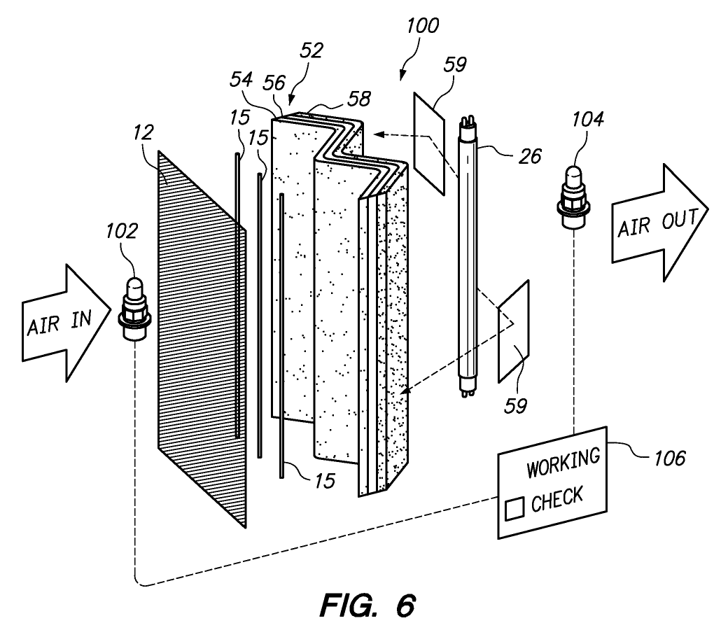

FIG. 6 is a perspective view of an air cleaner system 100 having gas sensors disposed in the air intake and air exhaust. The air cleaner system 100 includes the same elements as in system 50 of FIG. 4B, but it should be recognized that the gas sensors may be used in cooperation with any of the air cleaner systems described herein. Accordingly, an inlet gas sensor 102 measures one or more parameters of the inlet air and an outlet gas sensor 104 measures one or more parameters of the outlet air. The inlet and outlet gas sensors 102, 104 preferably measure the same parameters in order to facilitate a comparison. Each of the gas sensors 102, 104 communicate with a controller or indicator circuit 106 that indicates whether or not an appropriate change in the parameter has occurred between the air inlet and the air outlet. Various parameters could be measured or detected, but the sensors preferably detect oxygen, carbon monoxide, combustible gases, oxides of nitrogen, ammonia, hydrogen sulfide, and hydrogen.

It should be recognized that the air cleaners of the present invention may be implemented using one or more of the cleaning stages alone or in combination with any other of the one or more cleaning stages. For example, the light activated photocatalytic reactor 22 of FIG. 2 may be implemented as a stand alone unit in its own housing. While such a unit would not be intended to remove any significant amount of particulates from the air, the unit would still be effective at oxidizing VOCs, other unwanted or combustible vapors, or inactivating airborne pathogenic microorganisms. Furthermore, since conventional filtration units do not oxidize or inactivate these species, the light activated photocatalytic reactor would serve an important role. As a further example, the combination of a regenerable adsorption matrix with either a thermally activated porous oxidizing catalytic reactor or a light activated porous photocatalytic reactor, as shown in FIGS. 5A-5C, may provide considerable benefits by oxidizing VOCs, other unwanted or combustible vapors, and inactivating airborne pathogenic microorganisms, even in the absence of a prefilter or electrostatic precipitator.

The terms “comprising,” “including,” and “having,” as used in the claims and specification herein, shall be considered as indicating an open group that may include other elements not specified. The terms “a,” “an,” and the singular forms of words shall be taken to include the plural form of the same words, such that the terms mean that one or more of something is provided. The term “one” or “single” may be used to indicate that one and only one of something is intended. Similarly, other specific integer values, such as “two,” may be used when a specific number of things is intended. The terms “preferably,” “preferred,” “prefer,” “optionally,” “may,” and similar terms are used to indicate that an item, condition or step being referred to is an optional (not required) feature of the invention.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

What is claimed is:

1. An air cleaner, comprising:
a housing defining an airflow pathway between an air inlet and an air outlet;
a catalytic reactor having a catalyst secured on a porous substrate that is disposed transverse to the airflow pathway to cause air to flow through the porous substrate and into contact with the catalyst.

2. The air cleaner of claim 1, further comprising:
an adsorption matrix disposed transverse to the airflow pathway.

3. The air cleaner of claim 1, wherein the catalytic reactor includes a catalyst selected from the group consisting of a light activated oxidizing photocatalyst and a thermally activated oxidizing catalyst.

4. The air cleaner of claim 1, wherein the catalytic reactor includes a light source directed at a light activated oxidizing photocatalyst that is disposed on the porous substrate.

5. The air cleaner of claim 4, wherein the light source includes a bulb that produces light selected from ultraviolet light, visible light, and combinations thereof.

6. The air cleaner of claim 1, wherein the porous substrate is electrically grounded.

7. The air cleaner of claim 3, wherein the oxidizing photocatalyst comprises TiO₂ particles.

8. The air cleaner of claim 7, wherein the oxidizing photocatalyst comprises a binary oxide particle species selected from TiO₂/SiO₂, TiO₂/ZrO₂, TiO₂/SnO₂, TiO₂/WO₃, TiO₂/MoO₃, TiO₂/V₂O₅ and combinations thereof.

9. The air cleaner of claim 3, further comprising a catalyst disposed on the surfaces of the photocatalyst particles, wherein the catalyst is selected from the group consisting of a metal, metal oxide, or metal alloy, Pt group metals, Au group metals, Ir, Ru, Sn, Os, Mo, Zr, Cu, Nb, Rh, Pt—Sn, Pt—Mo, Pt—Ru, Ni—Zr, Pt—Rh, Pt—Ir, Pt—Ru—W, Pt—Ru—Os, Pt—Ru—Sn, Pt—Ni—Ti, Pt—Ni—Zr, Pt—Ni—Nb, platinum group metal oxides, gold group metal oxides, tin oxides, tungsten oxides, iridium oxides, rhodium oxides, ruthenium oxides and mixtures thereof.

10. The air cleaner of claim 9, wherein the catalyst disposed on the surfaces of the photocatalyst particles is a metal catalyst provided at a concentration or loading between about 0.01 wt % and about 5 wt %.

11. The air cleaner of claim 2, further comprising:
a heater in thermal communication with the adsorption matrix.

12. The air cleaner of claim 1, further comprising:
a particulate filter disposed upstream of the adsorption matrix and the catalytic reactor.

13. The air cleaner of claim 1, further comprising:
an electrostatic precipitator disposed upstream of the adsorption matrix and the catalytic reactor.

14. The air cleaner of claim 13, wherein the electrostatic precipitator comprises one or more discharge electrode and one of more collection electrode, wherein the one or more collection electrode is a macroporous member disposed transverse to the airflow pathway to cause air to flow through the macroporous member.

15. The air cleaner of claim 14, wherein the one or more collection electrode comprises two or more layers, wherein the two or more layers have openings that decrease in size from one layer to the next in the direction from the air inlet to the air outlet.

16. The air cleaner of claim 14, wherein the one or more discharge electrode is negatively charged and the one or more collection electrode is positively charged.

17. The air cleaner of claims 14, wherein the catalytic reactor includes a light source directed at a light activated oxidizing photocatalyst that is disposed on the porous substrate.

18. The air cleaner of claims 17, wherein the one or more collection electrode is secured in a unitary structure with the porous substrate of the catalytic reactor

19. The air cleaner of claim 18, wherein the porous substrate is electrically grounded.

20. The air cleaner of claim 18, wherein the photocatalyst is electrically insulated from the one or more collection electrode.

21. The air cleaner of claim 11, wherein the catalytic reactor includes a thermally activated oxidizing catalyst and a heater in thermal communication with the oxidizing catalyst, and wherein the catalytic reactor is disposed downstream of the adsorption matrix.

22. The air cleaner of claim 21, further comprising:
a thermoelectric heat exchanger disposed downstream of the catalytic reactor.

23. A method of removing volatile organic compounds from an air stream, comprising:
flowing the air stream through a porous adsorption matrix upstream of a porous oxidizing catalytic reactor, wherein the porous oxidizing catalytic reactor comprises an oxidizing catalyst surface;
adsorbing the volatile organic compounds from the air stream onto the porous adsorption matrix;
periodically heating the porous adsorption matrix to revolatilize the adsorbed compounds into the air stream;
flowing the air stream and revolatilized compounds into the oxidizing catalytic reactor; and
oxidizing the revolatilized compounds in the presence of an oxidant in the air stream at the oxidizing catalyst surface.

24. The method of claim 23, wherein the catalytic reactor includes a thermally activated oxidizing catalyst, the method further comprising:
heating the thermally activated oxidizing catalyst to a light off temperature substantially only immediately before and during the step of periodically heating the porous adsorption matrix.

25. The method of claim 24, further comprising:
allowing the thermally activated oxidizing catalyst to cool to ambient temperature in response to completing the step of periodically heating the porous adsorption matrix.

26. The method of claim 23, further comprising:
reducing the flow rate of the air stream through the catalytic reactor during the step of periodically heating to an air flow rate that is lower than the air flow rate during the step of adsorbing the volatile organic compounds.

27. The method of claim 23, wherein the catalytic reactor includes a light activated oxidizing catalyst, the method further comprising:
photogenerating radical species by directing light photons onto the surface of the oxidizing photocatalyst; and
oxidizing the volatile organic compounds in the air stream that contact the radical species.

28. The method of claim 27, further comprising:
inactivating microorganisms in the air stream that contact the radical species.

29. The method of claim 23, wherein the oxidizing catalyst surface includes a catalyst selected from the group consisting of a light activated photocatalyst, and a thermally activated catalyst.

30. The method of claim 23, wherein the oxidant source is selected from the group consisting of oxygen in air, ozone, vapor phase hydrogen peroxide, and combinations thereof.
